(19) United States
(12) Patent Application Publication
(10) Pub. No.: US 2025/0226092 A1
(43) Pub. Date: Jul. 10, 2025

(54) ELECTROCARDIOGRAM EVALUATION METHOD

(71) Applicant: NEC Corporation, Minato-ku, Tokyo (JP)

(72) Inventors: Yuan LUO, Tokyo (JP); Mitsuru NOMA, Tokyo (JP); Osamu HISAMATSU, Tokyo (JP); Akihiko SHIBANO, Tokyo (JP); Hiroaki KATAOKA, Tokyo (JP); Kosuke NISHIHARA, Tokyo (JP); Masahiro KUBO, Tokyo (JP)

(73) Assignee: NEC Corporation, Minato-ku, Tokyo (JP)

(21) Appl. No.: 18/849,592

(22) PCT Filed: Mar. 29, 2022
(86) PCT No.: PCT/JP2022/015450

§ 371 Date: Sep. 23, 2024

(51) Int. Cl. G16H 50/20; A61B 5/346; G16H 10/60

(52) U.S. Cl. CPC G16H 50/20; A61B 5/346; G16H 10/60

(57) ABSTRACT

An electrocardiogram evaluation apparatus of the present invention includes: an electrocardiogram acquiring unit that acquires electrocardiogram data measured from a person; an additional data acquiring unit that determines whether or not to acquire additional data to be used for evaluating the electrocardiogram data based on the electrocardiogram data, and acquires the additional data; and an evaluating unit that evaluates the electrocardiogram data based on the additional data. The electrocardiogram evaluation apparatus of the present invention can support decision-making by a medical professional, for example.

### TECHNICAL FIELD

The present invention relates to an electrocardiogram evaluation method, an electrocardiogram evaluation apparatus, and a program.

### BACKGROUND ART

One method for diagnosing a physical condition is to use an electrocardiogram. For example, in medical institutions, diagnosis of a physical condition is performed by measuring a 12-lead electrocardiogram or a monitored electrocardiogram with an electrocardiograph and evaluating the waveform of the electrocardiogram. Then, in recent years, as described in Patent Literature 1, an electrocardiogram is automatically analyzed and evaluated using a model generated through machine learning. For example, in Patent Literature 1, evaluation of an electrocardiogram is performed by generating a model by learning normal electrocardiograms and anomalous electrocardiograms of various diseases such as myocardial infarction, and inputting a measured electrocardiogram into the model.

### CITATION LIST

### Patent Literature

Patent literature 1: Japanese Unexamined Patent Application Publication No. JP-A 2020-130772

### SUMMARY OF INVENTION

### Technical Problem

However, even the electrocardiogram of a normal person may be determined to be that of a disease. Moreover, the waveforms of electrocardiograms may be indistinguishable between disease and noise. Consequently, a problem arises that it is difficult to accurately evaluate an electrocardiogram.

An object of the present invention is to provide an electrocardiogram evaluation method which can solve the abovementioned problem that it is difficult to accurately evaluate an electrocardiogram.

### Solution to Problem

An electrocardiogram evaluation method as an aspect of the present invention includes: acquiring electrocardiogram data measured from a person; determining whether or not to acquire additional data to be used for evaluating the electrocardiogram data based on the electrocardiogram data, and acquiring the additional data; and evaluating the electrocardiogram data based on the additional data.

Further, an electrocardiogram evaluation apparatus as an aspect of the present invention includes: an electrocardiogram acquiring unit that acquires electrocardiogram data measured from a person; an additional data acquiring unit that determines whether or not to acquire additional data to be used for evaluating the electrocardiogram data based on the electrocardiogram data, and acquires the additional data; and an evaluating unit that evaluates the electrocardiogram data based on the additional data.

Further, a computer program as an aspect of the present invention includes instructions for causing a computer to execute processes to: acquire electrocardiogram data measured from a person; determine whether or not to acquire additional data to be used for evaluating the electrocardiogram data based on the electrocardiogram data, and acquire the additional data; and evaluate the electrocardiogram data based on the additional data.

### Advantageous Effects of Invention

Configured as described above, the present invention enables accurate evaluation of an electrocardiogram.

### BRIEF DESCRIPTION OF DRAWINGS

FIG. 1 is a view showing the overall configuration of an information processing system in a first example embodiment of the present invention.

FIG. 2 is a block diagram showing the configuration of an electrocardiogram evaluation apparatus disclosed in FIG. 1.

FIG. 3 is a flowchart showing the operation of the electrocardiogram evaluation apparatus disclosed in FIG. 1.

FIG. 4 is a flowchart showing other operation of the electrocardiogram evaluation apparatus disclosed in FIG. 1.

FIG. 5 is a block diagram showing the hardware configuration of an electrocardiogram evaluation apparatus in a second example embodiment of the present invention.

FIG. 6 is a block diagram showing the configuration of the electrocardiogram evaluation apparatus in the second example embodiment of the present invention.

FIG. 7 is a flowchart showing the operation of the electrocardiogram evaluation apparatus in the second example embodiment of the present invention.

### DESCRIPTION OF EXAMPLE EMBODIMENTS

### First Example Embodiment

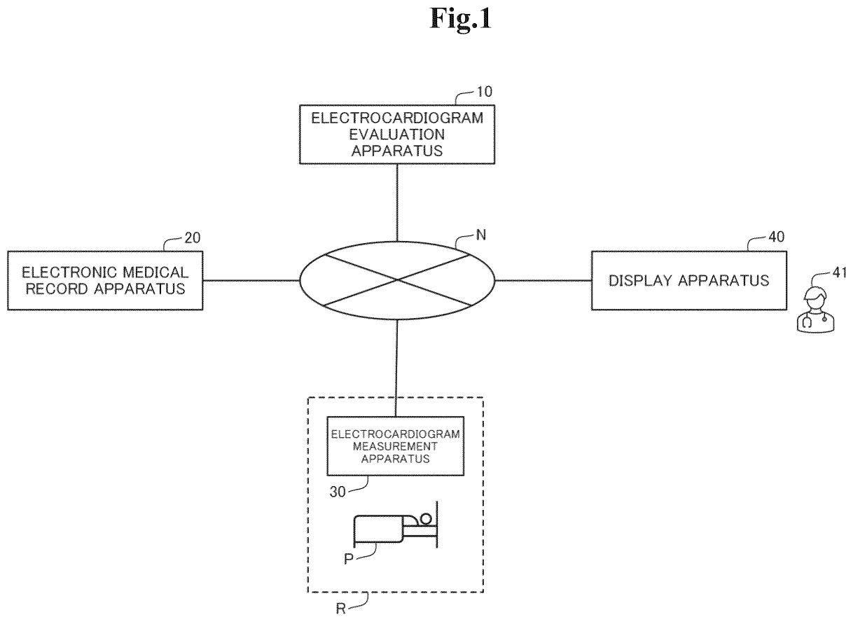

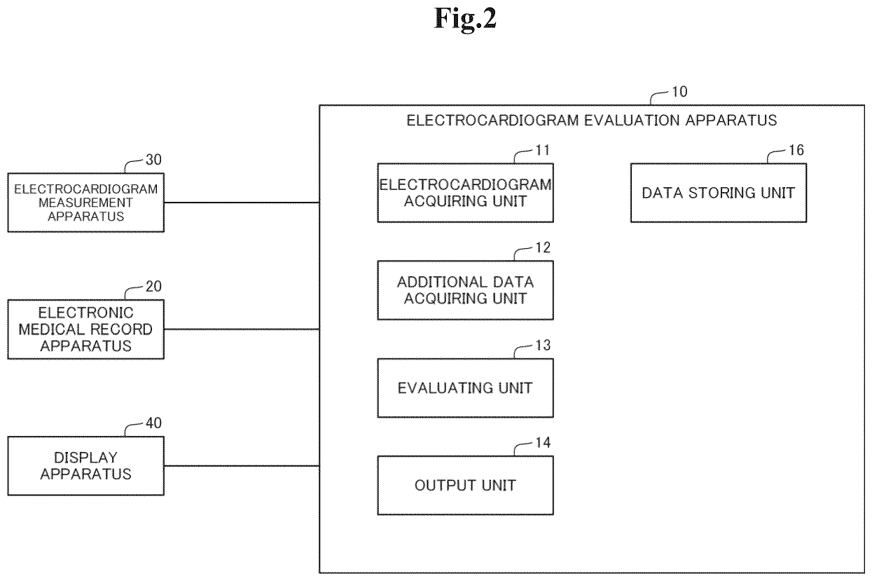

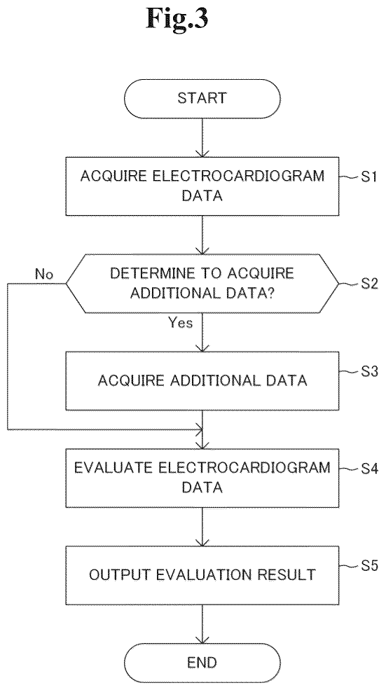

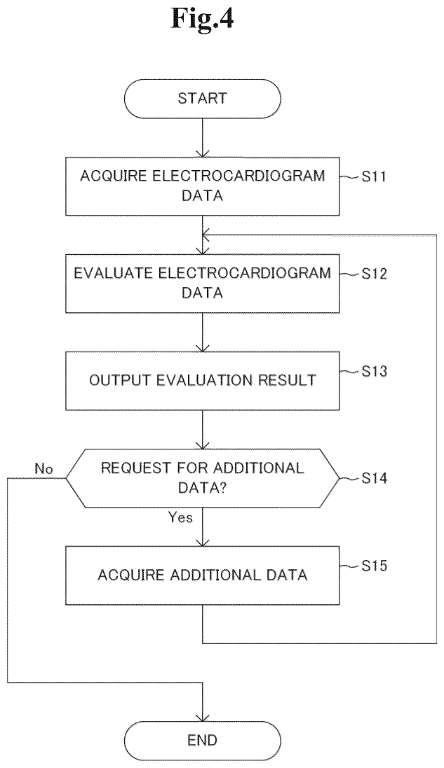

A first example embodiment of the present invention will be described with reference to FIGS. 1 to 4. FIGS. 1 and 2 are views for describing the configuration of an information processing system, and FIGS. 3 and 4 are views for describing the processing operation of the information processing system.

### [Configuration]

The information processing system of the present invention is for evaluating an electrocardiogram in order to diagnose a physical condition of a person in a medical institution. For example, the information processing system is for evaluating a normal condition and an anomalous condition such as myocardial infarction or subarachnoid hemorrhage from an electrocardiogram using a model generated through machine learning, and outputting the evaluation result to a medical professional such as a doctor.

As shown in FIG. 1, the information processing system includes an electrocardiogram evaluation apparatus 10, an electronic medical record apparatus 20, an electrocardiogram measurement apparatus 30, and a display apparatus 40, which are connected via a network N. The respective components will be described in detail below.

The electrocardiogram measurement apparatus 30 is an apparatus that measures an electrocardiogram from a person P. For example, the electrocardiogram measurement apparatus 30 is an electrocardiograph installed in a predetermined location R in a medical institution, such as a hospital room, an examination room and an intensive care unit, or a monitor electrocardiogram be worn by a person P, or a wearable device such as a wristwatch-type mobile terminal with an electrocardiograph incorporated in. In this example embodiment, it is installed in a medical institution and is the electrocardiogram measurement apparatus 30 that can measure a 12-lead electrocardiogram.

In addition to the configuration to measure electrocardiograms, the electrocardiogram measurement apparatus 30 also includes a configuration included by a general information processing apparatus, such as a communication device and an arithmetic logic unit, and also includes a function to transmit measured electrocardiogram data to the electronic medical record apparatus 20. Consequently, electrocardiogram data measured by the electrocardiogram measurement apparatus 30 is stored in an electronic medical record for each person P. As an example, an operator of the electrocardiogram measurement apparatus 30 specifies an electronic medical record of a person P who is a measurement target from among electronic medical records stored in the electronic medical record apparatus 20, and records the electrocardiogram data of the person P into the electronic medical record. In addition, in a case where the electrocardiogram measurement apparatus 30 is a wearable device, the electrocardiogram measurement apparatus 30 transmits electrocardiogram data to the electronic medical record apparatus 20 together with identification information of a person P and records the electrocardiogram data into an electronic medical record corresponding to the person P. However, electrocardiogram data may be recorded into an electronic medical record by any method.

Further, when recording electrocardiogram data into the electronic medical record apparatus 20, the electrocardiogram measurement apparatus 30 transmits identification information such as its own IP address in association with the electrocardiogram data to the electronic medical record apparatus 20. That is to say, the electrocardiogram measurement apparatus 30 transmits electrocardiogram data in association with identification information indicating the sender of the electrocardiogram data. At this time, identification information may be data that identifies a location in which the electrocardiogram measurement apparatus 30 as the sender of electrocardiogram data is installed, that is, a location of measurement of an electrocardiogram. For example, by previously assigning different identification information to the respective electrocardiogram measurement apparatuses 30 installed in locations R such as a hospital room, an examination room and an intensive care unit and storing correspondence information between the respective locations R and the respective identification information in the electronic medical record apparatus 20 or the electrocardiogram evaluation apparatus 10 to be described later, it is possible to identify a location of measurement of electrocardiogram data from identification information associated with the electrocardiogram data. Also, in a case where the electrocardiogram measurement apparatus 30 is a wearable device, associating identification information such as an IP address or information indicating a wearable device with electrocardiogram data makes it possible to identify that the electrocardiogram data has been measured by the wearable device.

In addition, electrocardiogram data measured by the electrocardiogram measurement apparatus 30 may be directly transmitted to the electrocardiogram evaluation apparatus 10.

The electronic medical record apparatus 20 is configured with a general information processing apparatus including an arithmetic logic unit and a memory unit managed by a medical institution, and stores an electronic medical record of a person P in the memory unit. For example, the examination result and the diagnosis result of a person P are recorded in an electronic medical record. As an example, the following data of a person P are recorded in an electronic medical record: basic physical data such as age, gender, height, and weight; measurement data such as heart rate, body temperature, blood pressure, and electrocardiogram data mentioned above; examination data such as a blood test result and an image diagnosis result; and medical condition data such as state of consciousness, current or past disease, condition at the time of diagnosis, and condition at the time of examination. The data in the electronic medical record is recorded by input of data by a diagnostician or an examiner, or by transmission of data from the electrocardiogram measurement apparatus 30 such as an electrocardiogram a wearable device mentioned above or from various examination devices or measurement devices.

At this time, the electrocardiogram data to be recorded in the electronic medical record is stored in association with information identifying a location of measurement of the electrocardiogram, such as the abovementioned identification information. Moreover, the electrocardiogram data is stored in association with data that represents circumstances at the time of measurement, for example, the course of events of measurement of the electrocardiogram. As an example, the data representing the course of events of measurement of the electrocardiogram includes a measurement taken when the person P visits the medical institution as a patient in a general outpatient or emergency outpatient unit, a measurement taken while the person P is hospitalized in a general ward or intensive care unit, and a measurement taken routinely using a wearable device.

The electrocardiogram evaluation apparatus 10 is configured with one or a plurality of information processing apparatuses each including an arithmetic logic unit and a memory unit. Then, as shown in FIG. 2, the electrocardiogram evaluation apparatus 10 includes an electrocardiogram acquiring unit 11, an additional data acquiring unit 12, an evaluating unit 13, and an output unit 14. The respective functions of the electrocardiogram acquiring unit 11, the additional data acquiring unit 12, the evaluating unit 13, and the output unit 14 can be realized by the arithmetic logic unit executing a program for realizing the respective functions stored in the memory unit. In addition, the electrocardiogram evaluation apparatus 10 includes a data storing unit 16. The data storing unit 16 is configured with the memory unit. The respective components will be described in detail below.

At the time of diagnosis of a person P, the electrocardiogram acquiring unit 11 acquires electrocardiogram data of a corresponding person P from the electronic medical record apparatus 20 described above. However, the electrocardiogram acquiring unit 11 may automatically acquire electrocardiogram data at a preset timing such as at a fixed time interval. In addition, the electrocardiogram acquiring unit 11 may acquire electrocardiogram data from the electrocardiogram measurement apparatus 30.

At the time of diagnosis of a person P, the addition data acquiring unit 12 determines whether or not to acquire additional data to be used for evaluation of electrocardiogram data. At this time, the additional data acquiring unit 12 determines whether or not to acquire additional data based on information associated with electrocardiogram data and the result of evaluation of electrocardiogram data.

For example, in a case where electrocardiogram data has been acquired from an electronic medical record and information of the electronic medical record corresponding to the electrocardiogram data is associated, additional data can be acquired from the electronic medical record, so that the additional data acquiring unit 12 determines to acquire the additional data. Moreover, in a case where electrocardiogram data is associated with identification information of the electrocardiogram measurement apparatus 30, which is data of the sender of the electrocardiogram data, an installation location of the electrocardiogram measurement apparatus 30 can be identified from the identification information, a measurement location of the electrocardiogram data can be identified from the installation location, and a record location of an electronic medical record, so that the additional data acquiring unit 16 determines to acquire additional data from the electronic medical record.

Further, the additional data acquiring unit 12 determines whether or not to acquire additional data based on the result of evaluation of electrocardiogram data by the evaluating unit 13 to be described later. For example, the additional data acquiring unit 12 calculates the accuracy of the evaluation result of electrocardiogram data, that is, the likelihood of the evaluation result, and determines to acquire additional data when the likelihood is lower than a set threshold value. For example, the additional data acquiring unit 12 calculates the likelihood using a likelihood function generated in advance based on statistics of the evaluation results. However, the additional data acquiring unit 12 may calculate the value of likelihood of the evaluation result by any method. In addition, the additional data acquiring unit 12 may determine to acquire additional data when an evaluation process by the evaluating unit 13 to be described later results in an error or when the value of the evaluation result falls outside a preset range, without calculating the likelihood of the evaluation result.

In addition, the additional data acquiring unit 12 may determine to acquire additional data in the case of receiving input of a request for acquisition of additional data from a medical professional such as a doctor after the evaluation result of the electrocardiogram data by the evaluating unit 13 to be described later is output. At this time, in a case where the request for acquisition of additional data includes information specifying the type of the additional data, the additional data acquiring unit 12 also accepts the information.

Then, when determining to acquire additional data as described above, the additional data acquiring unit 12 acquires the additional data and stores it into the data storing unit 16. At this time, the additional data acquiring unit 12, at the time of the abovementioned determination, identifies a location in which the electronic medical record of the person P is recorded from the information associated with the electrocardiogram data, and acquires data of the person P recorded in the electronic medical record from the electronic medical record apparatus 20. The additional data acquiring unit 12 acquires, as the additional data, basic physical data such as age, gender, height and weight, measurement data such as heart rate, body temperature and blood pressure, and medical condition data such as state of consciousness, current or past illnesses, condition at the time of diagnosis and condition at the time of examination, which are recorded in the electronic medical record. Furthermore, the additional data acquiring unit 12 may acquire electrocardiogram data of the person P from the electronic medical record as the additional data. At this time, the additional data acquiring unit 12 may acquire, as the electrocardiogram data, past data of the same type as the already acquired 12-lead electrocardiogram data to be evaluated, or may acquire data of a different type from 12-lead electrocardiogram data, for example, monitored electrocardiogram data. In addition, in a case where it is possible to acquire data of a monitored electrocardiogram currently worn by the person P, the additional data acquiring unit 12 may acquire the monitored electrocardiogram data.

Further, the additional data acquiring unit 12 may acquire remeasured electrocardiogram data. For example, in the case of determining that there is a need to remeasure the electrocardiogram data based on the evaluation result of the electrocardiogram data by the evaluating unit 13 to be described later, the additional data acquiring unit 12 displays a remeasurement request on the display apparatus 40 to a medical professional 41 via the output unit 14 to be described later, and acquires electrocardiogram data remeasured in response thereto as additional data. The remeasurement request displayed on the display apparatus 40 may include, for example, a notification of the state of electrocardiogram measurement and an advice on a remeasurement method to the medical professional 41.

In a case where the accepted additional data acquisition request includes information specifying the type of additional data, the additional data acquiring unit 12 acquires the specified type of additional data. For example, in a case where the additional data acquiring unit 12 is requested to acquire a disease of the person P, the course of events that the electrocardiogram has been taken, past electrocardiogram data, monitored electrocardiogram data, and the like, the additional data acquiring unit 12 acquires the data as additional data.

The evaluating unit 13 evaluates the electrocardiogram data based on the additional data acquired as described above. Specifically, as described above, the evaluating unit 13 evaluates the electrocardiogram data in consideration of the additional data, for example, a disease of the person P, the course of event that the electrocardiogram has been taken (whether or not it is an emergency), and past electrocardiogram data. As an example, the evaluating unit 13 compares the evaluation target electrocardiogram data with the past electrocardiogram data acquired as the additional data, and evaluates the waveform and detection value of the electrocardiogram data. Moreover, as another example, the evaluating unit 13 evaluates the electrocardiogram data of the person P using an evaluation model prepared in accordance with a disease of the person P or an emergency level. Here, an evaluation model is, for example, a model generated in advance through learning of anomalous electrocardiograms of various diseases, or an evaluation model according to the presence or absence of emergency. However, the evaluating unit 13 may evaluate the electrocardiogram data by any method.

Here, as examples of the evaluation result of electrocardiogram data, the evaluating unit 13 may calculate the physical anomaly degree of the person P, the emergency level of treatment for the person P, and the presence or absence of a specific disease. However, the evaluating unit 13 may perform evaluation of any content as long as the content can be calculated from electrocardiogram data.

In addition, the evaluating unit 13 may evaluate electrocardiogram data a plurality of times while changing additional data to be considered. Moreover, the evaluating unit 13 may evaluate electrocardiogram data without considering additional data. For example, the evaluating unit 13 may first evaluate target electrocardiogram data before acquiring additional data.

The output unit 14 outputs the evaluation result by the evaluating unit 13 to the display apparatus 40. At this time, the output unit 14 may output additional data considered at the time of evaluation by the evaluating unit 13 together with the evaluation result, or may output additional data of the person P regardless of whether or not it has been considered at the time of evaluation. Before acquiring additional data, the output unit 14 may output only the evaluation result of electrocardiogram data. The evaluation result and additional data thus displayed may also serve as an advice to the medical professional 41. For example, the output unit 14 displays the physical anomaly degree of the person P, the emergency level of treatment for the person P, the presence or absence of a specific disease and the like mentioned above, as the evaluation result of electrocardiogram data, which enables the medical professional 41 to grasp the condition of the person P and will be useful for subsequent treatment and additional examinations and measurements.

Further, in a case where a request for acquisition of additional data is input from the medical professional 41 such as a doctor after the evaluation result is output to the display apparatus 40, the output unit 14 accepts the request for acquisition of additional data. At this time, in a case where the request for acquisition of additional data includes information specifying the type of additional data, the output unit 14 also accepts the information. Then, the output unit 14 notifies the additional data acquiring unit 12 of the accepted acquisition request.

### [Operation]

Next, the operation of the above electrocardiogram evaluation apparatus 10 will be described first with reference to a flowchart of FIG. 3.

First, at the time of diagnosis of a person P, the electrocardiogram evaluation apparatus 10 acquires electrocardiogram data of the person P (step S1). At this time, the electrocardiogram evaluation apparatus 10 acquires 12-lead electrocardiogram data.

Next, the electrocardiogram evaluation apparatus 10 determines whether or not to acquire addition data to be used for evaluating the electrocardiogram data (step S2). Here, the electrocardiogram evaluation apparatus 10 determines whether or not to acquire additional data based on information associated with the electrocardiogram data. For example, in a case where the electrocardiogram data is associated with information of an electronic medical record from which the electrocardiogram data has been acquired, or in a case where the electrocardiogram data is associated with identification information of the electrocardiogram measurement apparatus 30 that is the sender of the electrocardiogram data, additional data can be acquired from a storage location of the electrocardiogram data, such as the electronic medical record, so that the electrocardiogram evaluation apparatus 10 determines to acquire additional data (Yes at step S2). Then, the electrocardiogram evaluation apparatus 10 acquires additional data from the electronic medical record, or the like (step S3). For example, the electrocardiogram evaluation apparatus 10 acquires record data, past electrocardiogram data and monitored electrocardiogram data that is of another type of the person P recorded in the electronic medical record.

Next, the electrocardiogram evaluation apparatus 10 evaluates the electrocardiogram data based on the acquired additional data (step S4). Specifically, in the case of acquiring the disease of the person P, the course of events that the electrocardiogram has been taken (whether or not it an emergency) and past electrocardiogram data as the additional data, the electrocardiogram evaluation apparatus 10 considers them to evaluate the electrocardiogram data. As an example, the electrocardiogram evaluation apparatus 10 compares the evaluation target electrocardiogram data with past electrocardiogram data acquired as the additional data, and evaluates the waveform and detection value of the electrocardiogram data. Moreover, as another example, the electrocardiogram evaluation apparatus 10 evaluates the electrocardiogram data of the person P using an evaluation model prepared in accordance with the disease of the person P and an emergency level.

After that, the electrocardiogram evaluation apparatus 10 outputs the result of evaluation of the electrocardiogram data to the display apparatus 40 (step S5). At this time, the electrocardiogram evaluation apparatus 10 also outputs the additional data considered at the time of evaluation together with the evaluation result.

As described above, according to this example embodiment, the electrocardiogram data is evaluated using the vital data, past electrocardiogram data, other electrocardiogram data and the like of the person P in addition to the electrocardiogram data of the person P. Thus, since the electrocardiogram data is evaluated in consideration of various data, it is possible to evaluate the electrocardiogram with high accuracy even when the waveforms of electrocardiogram data are similar and difficult to distinguish between different physical conditions of the person P. For example, it will be possible to prevent a normal person from being mistaken for a person with a disease, and to distinguish between the waveform of disease and the waveform of noise.

Next, another example of the operation of the electrocardiogram evaluation apparatus 10 will be described with reference to the flowchart of FIG. 4.

First, at the time of diagnosis of a person P, the electrocardiogram evaluation apparatus 10 acquires electrocardiogram data of the person P (step S11). At this time, the electrocardiogram evaluation apparatus 10 acquires 12-lead electrocardiogram data. Then, the electrocardiogram evaluation apparatus 10 evaluates the acquired electrocardiogram data (step S12). That is to say, unlike in the case of FIG. 3, the electrocardiogram evaluation apparatus 10 first evaluates the electrocardiogram data without considering additional data in this example. For example, the electrocardiogram evaluation apparatus 10 evaluates the electrocardiogram data of the person P by evaluating the waveform or detection value of the electrocardiogram data or by using an evaluation model.

Next, the electrocardiogram evaluation apparatus 10 outputs the evaluation result of the electrocardiogram data to the display apparatus 40 (step S13). This allows the medical professional 41 and so forth to see the evaluation result of the electrocardiogram data on the display apparatus 40. Here, in a case where the medical professional 41 who has seen the evaluation result of the electrocardiogram data wishes to obtain evaluation of the electrocardiogram data in consideration of the additional data, the medical professional 41 requests acquisition of additional data from the display apparatus 40. At this time, the medical professional 41 requests acquisition of additional data, including information specifying the type of additional data. Consequently, when accepting a request for acquisition of additional data from the display apparatus 40, the electrocardiogram evaluation apparatus 10 determines to acquire additional data (Yes at step S14).

Next, the electrocardiogram evaluation apparatus 10 acquires additional data from an electronic medical record or the like (step S15). In a case where the accepted additional data acquisition request includes information specifying the type of additional data, the electrocardiogram evaluation apparatus 10 acquires the specified type of additional data.

Next, the electrocardiogram evaluation apparatus 10 evaluates the electrocardiogram data based on the acquired additional data (step S12). As an example, similar to the evaluation method described in FIG. 3, in the case of acquiring the disease of the person P, the course of events that the electrocardiogram has been taken (whether or not it is an emergency) and past electrocardiogram data as additional data, the electrocardiogram evaluation apparatus 10 considers them to evaluate the electrocardiogram data.

Next, the electrocardiogram evaluation apparatus 10 outputs the result of evaluation of the electrocardiogram data to the display apparatus 40 (step S13). At this time, the electrocardiogram evaluation apparatus 10 outputs, together with the evaluation result, the additional data considered at the time of evaluation. This allows the medical professional 41 and so forth to see the evaluation result of the electrocardiogram data evaluated again in consideration of the additional data on the display apparatus 40.

Next, in a case where the medical professional 41 who has seen the evaluation result of the electrocardiogram data wishes to obtain the evaluation of the electrocardiogram data in consideration of still other additional data, the medical professional 41 requests acquisition of additional data from the display apparatus 40, including information specifying the type of the still other additional data. Consequently, when accepting a request for acquisition of other additional data from the display apparatus 40, the electrocardiogram evaluation apparatus 10 determines to acquire the additional data (Yes at step S14).

After that, in the same manner as described above, the electrocardiogram evaluation apparatus 10 acquires other additional data from the electronic medical record or the like (step S15), and evaluates the electrocardiogram data based on the acquired other additional data (step S12).

As described above, according to this example embodiment, it is possible to further request additional data in accordance with the evaluation result of electrocardiogram data, and it is possible to obtain an evaluation result in consideration of desired additional data. As a result, it is possible to further accurately evaluate an electrocardiogram.

### Second Example Embodiment

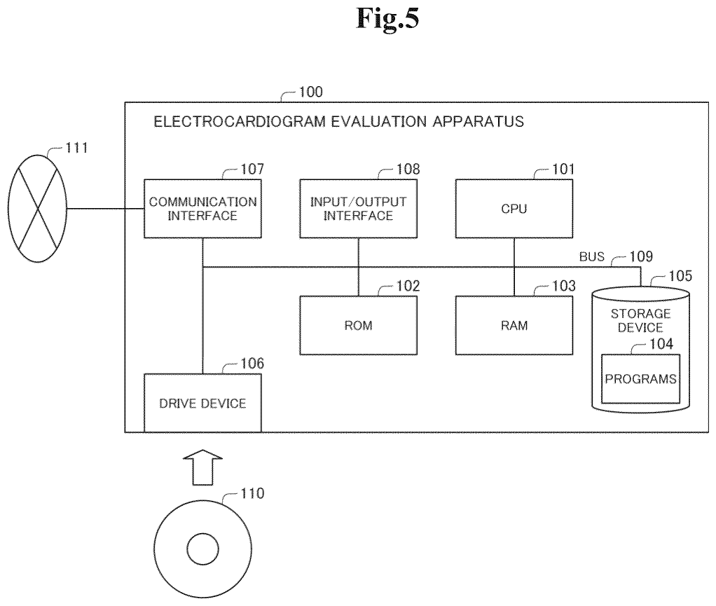

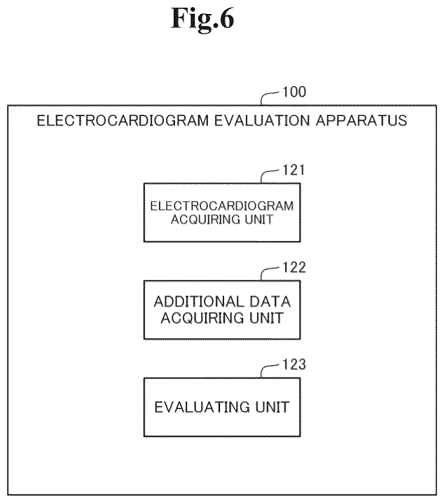

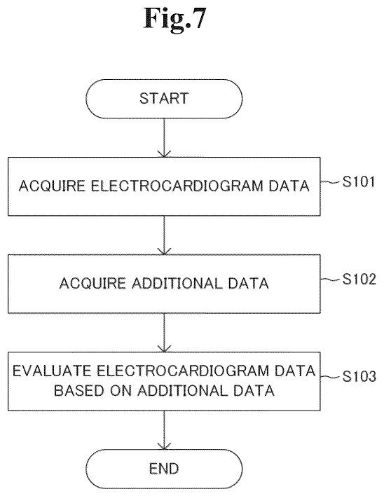

Next, a second example embodiment of the present invention will be described with reference to FIGS. 5 to 7. FIGS. 5 and 6 are block diagrams showing the configuration of an electrocardiogram evaluation apparatus in the second example embodiment, and FIG. 7 is a flowchart showing the operation of the electrocardiogram evaluation apparatus. In this example embodiment, the overview of the configurations of the electrocardiogram evaluation apparatus and the electrocardiogram evaluation method described in the above example embodiment is shown.

First, with reference to FIG. 5, the hardware configuration of an electrocardiogram evaluation apparatus 100 in this example embodiment will be described. The electrocardiogram evaluation apparatus 100 is configured with a general information processing apparatus, and as an example, has the following hardware configuration including:
a CPU (Central Processing Unit) 101 (arithmetic logic unit) (or a GPU (Graphics Processing Unit));
a ROM (Read Only Memory) 102 (memory unit);
a RAM (Random Access Memory) 103 (memory unit);
programs 104 loaded to the RAM 103;
a storage apparatus 105 storing the programs 104;
a drive apparatus 106 that reads from and writes into a storage medium 110 outside the information processing apparatus;
a communication interface 107 connected to a communication network 111 outside the information processing apparatus;
an input/output interface 108 that inputs and outputs data; and
a bus 109 that connects the respective components.

Then, the electrocardiogram evaluation apparatus 100 can construct and include an electrocardiogram acquiring unit 121, an additional data acquiring unit 122, and an evaluating unit 123 shown in FIG. 6 by acquisition and execution of the programs 104 by the CPU 101. The programs 104 are, for example, stored in advance in the storage apparatus 105 or the ROM 102, and are loaded into the RAM 103 and executed by the CPU 101 as necessary. The programs 104 may be provided to the CPU 101 via the communication network 111, or the programs 104 may be stored in the storage medium 110 in advance and read out by the drive device 106 and provided to the CPU 101. However, the electrocardiogram acquiring unit 121, the additional data acquiring unit 122, and the evaluating unit 123 described above may be constructed using dedicated electronic circuits for realizing such means.

FIG. 5 shows an example of the hardware configuration of the information processing apparatus serving as the electrocardiogram evaluation apparatus 100, and the hardware configuration of the information processing apparatus is not limited to the above case. For example, the information processing apparatus may be configured with part of the above configuration, such as without the drive apparatus 106.

Then, the electrocardiogram evaluation apparatus 100 executes an electrocardiogram evaluation method shown in the flowchart of FIG. 7 by the functions of the electrocardiogram acquiring unit 121, the additional data acquiring unit 122, and the evaluating unit 123 constructed by the program as described above.

As shown in FIG. 7, the electrocardiogram evaluation apparatus 100 executes processes to:
acquire electrocardiogram data measured from a person (step S101);
determine whether or not to acquire additional data to be used for evaluating the electrocardiogram data based on the electrocardiogram data, and acquire the additional data (step S102); and
evaluate the electrocardiogram data based on the additional data (step S103).

Configured as described above, the present invention allows evaluation of electrocardiogram data of a person P using, in additional to the electrocardiogram data, vital data, past electrocardiogram data, other electrocardiogram data and the like of the person P. Since electrocardiogram data is evaluated in consideration of various data, it is possible to accurately evaluate an electrocardiogram.

The abovementioned programs can be stored and provided to a computer using various types of non-transitory computer-readable mediums. Non-transitory computer-readable mediums include various types of tangible storage mediums. Examples of non-transitory computer-readable mediums include a magnetic recording medium (e.g., floppy disk, magnetic tape, hard disk drive), a magneto-optical recording medium (e.g., magneto-optical disk), a CD-ROM (Read Only Memory), a CD-R, a CD-R/W, and a semiconductor memory (e.g., mask ROM, PROM (Programmable ROM), EPROM (Erasable PROM), flash ROM, and RAM (Random Access Memory)). The program may also be provided to a computer by various types of transitory computer-readable mediums. Examples of transitory computer-readable mediums include an electric signal, an optical signal, and an electromagnetic wave. The temporary computer-readable medium can provide the program to the computer via a wired communication path such as an electric wire or an optical fiber, or via a wireless communication path.

Although the present invention has been described above with reference to the above example embodiments, the present invention is not limited to the above example embodiments. The configuration and details of the present invention can be modified in various ways that are understandable to a person skilled in the art within the scope of the present invention. Moreover, at least one or more of the functions of the electrocardiogram acquiring unit 121, the additional data acquiring unit 122 and the evaluating unit 123 described above may be executed by an information processing apparatus installed and connected anywhere on the network, that is, may be executed by so-called cloud computing.

### Supplementary Notes

The whole or part of the example embodiments disclosed above can be described as the following supplementary notes. The overview of the configurations of an electrocardiogram evaluation method, an electrocardiogram evaluation apparatus, and a program according to the present invention will be described below. However, the present invention is not limited to the following configurations.

### Supplementary Note 1

An electrocardiogram evaluation method comprising:
acquiring electrocardiogram data measured from a person;
determining whether or not to acquire additional data to be used for evaluating the electrocardiogram data based on the electrocardiogram data, and acquiring the additional data; and
evaluating the electrocardiogram data based on the additional data.

### Supplementary Note 2

The electrocardiogram evaluation method according to Supplementary Note 1, comprising
acquiring the additional data from an electronic medical record of the person.

### Supplementary Note 3

The electrocardiogram evaluation method according to Supplementary Note 2, comprising
acquiring past electrocardiogram data of the person from the electronic medical record of the person, as the additional data.

### Supplementary Note 4

The electrocardiogram evaluation method according to any one of Supplementary Notes 1 to 3, comprising
acquiring the additional data based on data representing a sender of the electrocardiogram data.

### Supplementary Note 5

The electrocardiogram evaluation method according to any one of Supplementary Notes 1 to 4, comprising:
outputting a result of the evaluation of the electrocardiogram data; and
accepting a request for the additional data input in accordance with the output of the result of the evaluation, and acquiring the additional data.

### Supplementary Note 6

The electrocardiogram evaluation method according to any one of Supplementary Notes 1 to 4, comprising:
outputting a result of the evaluation of the electrocardiogram data and also information based on the additional data; and
accepting a request for the additional data input in accordance with the output of the result of the evaluation, and acquiring the additional data.

### Supplementary Note 7

The electrocardiogram evaluation method according to any one of Supplementary Notes 1 to 6, comprising:
acquiring 12-lead electrocardiogram data;
evaluating the 12-lead electrocardiogram data;
acquiring monitored electrocardiogram data in accordance with a result of the evaluation of the 12-lead electrocardiogram data; and
evaluating using the 12-lead electrocardiogram data and the monitored electrocardiogram data.

### Supplementary Note 8

The electrocardiogram evaluation method according to any one of Supplementary Notes 1 to 7, comprising:
evaluating the electrocardiogram data and also calculating a likelihood of a result of the evaluation; and
acquiring the additional data in accordance with a value of the likelihood.

### Supplementary Note 9

An electrocardiogram evaluation apparatus comprising:
an electrocardiogram acquiring unit that acquires electrocardiogram data measured from a person;
an additional data acquiring unit that determines whether or not to acquire additional data to be used for evaluating the electrocardiogram data based on the electrocardiogram data, and acquires the additional data; and
an evaluating unit that evaluates the electrocardiogram data based on the additional data.

### Supplementary Note 10

The electrocardiogram evaluation apparatus according to Supplementary Note 9, wherein
the additional data acquiring unit acquires the additional data from an electronic medical record of the person.

### Supplementary Note 11

The electrocardiogram evaluation apparatus according to Supplementary Note 10, wherein
the additional data acquiring unit acquires past electrocardiogram data of the person from the electronic medical record of the person, as the additional data.

### Supplementary Note 12

The electrocardiogram evaluation apparatus according to any one of Supplementary Notes 9 to 11, wherein
the additional data acquiring unit acquires the additional data based on data representing a sender of the electrocardiogram data.

### Supplementary Note 13

The electrocardiogram evaluation apparatus according to any one of Supplementary Notes 9 to 12, wherein:
the evaluating unit outputs a result of the evaluation of the electrocardiogram data; and
the additional data acquiring unit accepts a request for the additional data input in accordance with the output of the result of the evaluation, and acquires the additional data.

### Supplementary Note 14

The electrocardiogram evaluation apparatus according to any one of Supplementary Notes 9 to 12, wherein:
the evaluating unit outputs a result of the evaluation of the electrocardiogram data and also information based on the additional data; and
the additional data acquiring unit accepts a request for the additional data input in accordance with the output of the result of the evaluation, and acquires the additional data.

### Supplementary Note 15

The electrocardiogram evaluation apparatus according to any one of Supplementary Notes 9 to 14, wherein:
the electrocardiogram acquiring unit acquires 12-lead electrocardiogram data;
the evaluating unit evaluates the 12-lead electrocardiogram data;
the additional data acquiring unit acquires monitored electrocardiogram data in accordance with a result of the evaluation of the 12-lead electrocardiogram data; and
the evaluating unit evaluates using the 12-lead electrocardiogram data and the monitored electrocardiogram data.

### Supplementary Note 16

The electrocardiogram evaluation apparatus according to any one of Supplementary Notes 9 to 15, wherein:
the evaluating unit evaluates the electrocardiogram data and also calculates a likelihood of a result of the evaluation; and
the additional data acquiring unit acquires the additional data in accordance with a value of the likelihood.

### Supplementary Note 17

A non-transitory computer-readable storage medium storing a computer program, the computer program comprising instructions for causing a computer to execute processes to:
acquire electrocardiogram data measured from a person;
determine whether or not to acquire additional data to be used for evaluating the electrocardiogram data based on the electrocardiogram data, and acquire the additional data; and
evaluate the electrocardiogram data based on the additional data.

### REFERENCE SIGNS LIST

10 electrocardiogram evaluation apparatus
11 electrocardiogram acquiring unit
12 additional data acquiring unit
13 evaluating unit
14 output unit
16 data storing unit
20 electronic medical record apparatus
30 electrocardiogram measurement apparatus
40 display apparatus
P person
100 electrocardiogram evaluation apparatus
101 CPU
102 ROM
103 RAM
104 programs
105 storage device
106 drive device
107 communication interface
108 input/output interface
109 bus
110 storage medium
111 communication network
121 electrocardiogram acquiring unit
122 additional data acquiring unit
123 evaluating unit

What is claimed is:

1. An electrocardiogram evaluation method comprising:
acquiring electrocardiogram data measured from a person;
determining whether or not to acquire additional data to be used for evaluating the electrocardiogram data based on the electrocardiogram data, and acquiring the additional data; and
evaluating the electrocardiogram data based on the additional data.

2. The electrocardiogram evaluation method according to claim 1, comprising
acquiring the additional data from an electronic medical record of the person.

3. The electrocardiogram evaluation method according to claim 2, comprising
acquiring past electrocardiogram data of the person from the electronic medical record of the person, as the additional data.

4. The electrocardiogram evaluation method according to claim 1, comprising
acquiring the additional data based on data representing a sender of the electrocardiogram data.

5. The electrocardiogram evaluation method according to claim 1, comprising:
outputting a result of the evaluation of the electrocardiogram data; and
accepting a request for the additional data input in accordance with the output of the result of the evaluation, and acquiring the additional data.

6. The electrocardiogram evaluation method according to claim 1, comprising:
outputting a result of the evaluation of the electrocardiogram data and also information based on the additional data; and
accepting a request for the additional data input in accordance with the output of the result of the evaluation, and acquiring the additional data.

7. The electrocardiogram evaluation method according to claim 1, comprising:
acquiring 12-lead electrocardiogram data;
evaluating the 12-lead electrocardiogram data;
acquiring monitored electrocardiogram data in accordance with a result of the evaluation of the 12-lead electrocardiogram data; and
evaluating using the 12-lead electrocardiogram data and the monitored electrocardiogram data.

8. The electrocardiogram evaluation method according to claim 1, comprising:
evaluating the electrocardiogram data and also calculating a likelihood of a result of the evaluation; and
acquiring the additional data in accordance with a value of the likelihood.

9. An electrocardiogram evaluation apparatus comprising:
at least one memory storing processing instructions; and
at least one processor configured to execute the processing instructions to:
acquire electrocardiogram data measured from a person;
determine whether or not to acquire additional data to be used for evaluating the electrocardiogram data based on the electrocardiogram data, and acquire the additional data; and
evaluate the electrocardiogram data based on the additional data.

10-16. (canceled)

17. A non-transitory computer-readable storage medium storing a computer program, the computer program comprising instructions for causing a computer to execute processes to:
acquire electrocardiogram data measured from a person;
determine whether or not to acquire additional data to be used for evaluating the electrocardiogram data based on the electrocardiogram data, and acquire the additional data; and
evaluate the electrocardiogram data based on the additional data.

18. The electrocardiogram evaluation method according to claim 1, comprising
evaluating the electrocardiogram data based on the additional data using an evaluation model generated through machine learning.
